(19) United States
(12) Patent Application Publication
(10) Pub. No.: US 2025/0144269 A1
(43) Pub. Date: May 8, 2025

(54) A protein composition having a joint repair function and its preparation method and application

(71) Applicant: BEIJING DARWIN BIOTECH CO., LTD., Beijing (CN)

(72) Inventors: Yu WANG, Beijing (CN); Wenyong GAO, Beijing (CN); Lin CHEN, Beijing (CN); Jianjun LI, Beijing (CN)

(73) Assignee: BEIJING DARWIN BIOTECH CO., LTD., Beijing (CN)

(21) Appl. No.: 18/833,860

(22) PCT Filed: Jan. 28, 2023
(86) PCT No.: PCT/CN2023/073596

§ 371 Date: Jul. 26, 2024

(30) Foreign Application Priority Data
Jan. 28, 2022 (CN) 202210105729.X
Apr. 2, 2022 (CN) 202210351455.2
Jul. 5, 2022 (CN) 202210793711.3

(51) Int. Cl. A61L 27/22; A61P 19/00; C07K 1/16; C12N 5/0775; C12P 21/00

(52) U.S. Cl. CPC A61L 27/22; A61P 19/00; C07K 1/16; C12N 5/0668; C12P 21/00; A61L 2430/24; C12N 2500/25; C12N 2500/32; C12N 2501/11; C12N 2501/115; C12N 2501/999

(57) ABSTRACT

The present disclosure relates to a protein composition having a joint repair function. Preparation of the protein composition comprises the following steps: adding any one of or a combination of a nuclease or Benzonase nuclease of 20 U/mL-35 U/mL into a cell protein extract, placing same at 37° C.±1° C. for enzymatic hydrolysis for 15-40 minutes, and separating and purifying the prepared enzymatic hydrolysate to obtain the protein composition. The protein composition obtained in the present disclosure has the effects of cell repair, joint repair, or cartilage repair, and is used for preventing and treating any one of or a complication of traumatic arthropathy, degenerative osteoarthropathy, joint injury, refractory wound lesions, knee osteoarthrosis, or cartilage injury.

### TECHNICAL FIELD

The present disclosure belongs to the field of biopharmaceutical technology and specifically relates to a protein composition having a joint repair function, its preparation method, and its application.

### BACKGROUND TECHNOLOGY

Arthritis is known as an incurable cancer, with a high disability rate and a serious impact on quality of life. Joint diseases caused by degenerative diseases and sports injuries have exceeded 150 million people in China. It is predicted that the Chinese joint and sports medicine market alone will exceed 20 billion CNY per year and grow at a rate of over 20%. The survey on the prevalence of primary osteoarthritis in the population aged 40 and above in China shows that the incidence rate of primary osteoarthritis in the population aged 40 to 70 is 62.2% and shows an age correlation. According to national statistical data, the population aged 65 and above in China is expected to exceed 210 million by 2025 (accounting for about 15%), and problems such as joint injuries, degenerative diseases, or cartilage injuries among the elderly are increasing year by year. Joint injuries (such as traumatic joint lesions, degenerative bone and joint lesions, etc.) are common clinical conditions, including refractory wound lesions, knee osteoarthritis, knee cartilage wear, cartilage injuries, etc.

The treatment or repair methods of joint injury and articular cartilage injury include anti-inflammatory, analgesia (pain relief), physical therapy, acupuncture and moxibustion, joint replacement, stem cell treatment, radiofrequency ablation, chondrocyte transplantation, osteochondral transplantation, engineered cartilage, such as physical therapy and nutrition represented by traditional Chinese medicine physical therapy and aminosugar, lubrication and relief represented by chitosan and hyaluronic acid, anti-inflammatory treatment represented by PRP, and joint replacement represented by arthroscopy and artificial joints. Except for joint replacement, all of them focus on alleviating symptoms and delaying the course of disease, and lack of truly effective and safe treatment methods.

Joint cartilage lacks the distribution of blood vessels, nerves, and lymphatic systems, and contains a small amount of chondrocytes with limited migration and proliferation ability, making it difficult for joint cartilage damage to self heal. When there is a malfunction in the blood supply and nerve innervation of the articular cartilage, it is easy to cause joint damage, which in turn affects the joint repair ability. If timely and effective treatment and repair of the injury are not carried out, the injury will continue to worsen and cause osteoarthritis, leading to joint pain, joint swelling or movement disorders, and even irreversible pathological changes and joint degenerative changes, affecting the patient's movement and disability. Joint injury repair is a long-term, gradual, and slow process. Timely repair of damaged cells can significantly improve and treat related lesions.

When cells and microorganisms are subjected to external stimuli and external stressors (including cold, heat, acid, alkali, current, radiation, chemicals, etc.), they will be induced to produce stress proteins due to stress responses. Reference 1 (New limonophyllines A-C from the stem of Atalantia monophylla and cytotoxicity against cholangiocarcinoma and HepG2 cell lines, Arch. Pharm. Res. (2018) 41:431-437) reveals that compound 1-16 extracted from the Rutaceae plant (Atalantia monophylla) exhibits activities such as inhibiting tumor cell growth.

Mesenchymal stem cells (MSCs) have the potential for self replication and multi-lineage differentiation, and they are widely present in tissues such as bone marrow, fat, synovium, dental pulp, amniotic fluid, placenta, umbilical cord, embryo, umbilical cord blood, amniotic membrane, peripheral blood, muscle, urine, etc. They have the characteristics of wide sources, no need for matching, low infection rate, strong differentiation potential, strong proliferation ability, and easy collection. They can produce stem cell growth factor (SCF), nerve growth factor (NGF), interleukin-6 (IL-6), interleukin-7 (IL-7), tumor necrosis factor (TNF), interferon (IFN), etc. Active factors are involved in regulating cell growth, apoptosis, differentiation, antiviral therapy, immune maturation, and other processes. They can be used for immune regulation, tissue repair, and the treatment of acute lung injury, severe pneumonia, acute respiratory distress syndrome, and other diseases. However, MSCs products need to be refrigerated in their production, storage, transportation, and application processes, and their cell viability is only maintained for ≤12 hours, which limits their therapeutic applications. Therefore, it is necessary to develop safe and effective joint repair drugs to meet clinical needs.

### SUMMARY

The purpose of the present disclosure is to provide a protein composition having a joint repair function, comprising the following steps for preparation:

(1) Add any one of or a combination of a nuclease or an omnipotent nuclease of 20 U/mL-35 U/mL into a cell protein extract of the present disclosure, and place it under 37° C.±1° C. for enzymatic hydrolysis for 15-40 minutes to obtain the enzymatic hydrolysate, then place the enzymatic hydrolysate under 2° C.-4° C. conditions for backup;

(2) Under the conditions of 2° C.-8° C., the enzymatic hydrolysate prepared in step (1) was prepared with an eluent at a concentration of 5-15 mg/mL, and then passed through a chromatographic column with an eluent flow rate of 0.1-1 mL/min. An eluent fraction with a UV wavelength of 280 nm is monitored and collected. The eluent is composed of 50 mmol/L phosphate buffer (pH 6.8) containing 300 mmol/L sodium chloride.

In the preferred technical solution of the present disclosure, the preparation of the cell protein extract comprises the following steps:

S-1: Place mesenchymal passaged cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acid (18AA) 0.01-0.1%, and 2-10 μmol/L of a stressor, and then culture it under conditions of 37.0° C.±0.5° C. and 5%±1.0% CO₂ for 10 to 14 hours. The cells undergo separation, are separated, washed, and collected. The stressors are selected from any one of compounds 1-16 or a combination thereof;

| Compounds | General formula | Substituents |
|---|---|---|
| 1 2 3 | 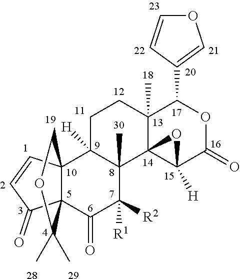 | R¹ = OCH₃, R² = OH R¹ = R² = OH R¹ = OH, R² = H |
| 4 5 | 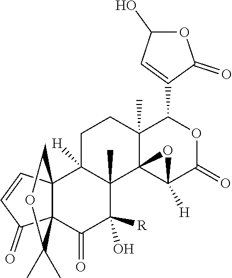 | R = H R = OH |
| 6 7 8 9 10 11 12 | 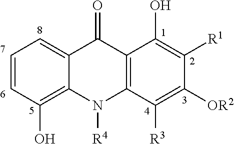 | R¹ = prenyl, R² = H, R³ = prenyl, R⁴ = CH₃ R¹ = prenyl, R² = H, R³ = prenyl, R⁴ = H R¹ = H, R² = H, R³ = OCH₃, R⁴ = CH₃ R¹ = H, R² = CH₃, R³ = OCH₃, R⁴ = CH₃ R¹ = OCH₃, R² = H, R³ = H, R⁴ = CH₃ R¹ = OCH₃, R² = H, R³ = OCH₃, R⁴ = CH₃ R¹ = prenyl, R² = CH₃, R³ = H, R⁴ = H |
| 13 14 | 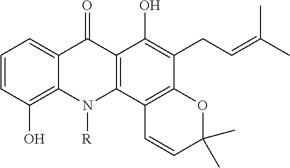 | R = H R = CH₃ |
| 15 | 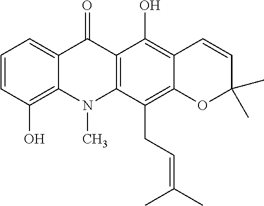 [SMILES: CC(C)=CCc1c2c(c(O)c3c(=O)c4cccc(O)c4n(C)c13)C=CC(C)(C)O2] | |
| 16 | 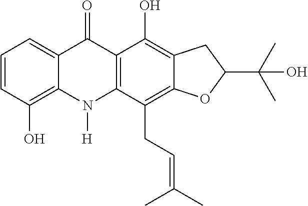 [SMILES: CC(C)=CCc1c2c(c(O)c3c(=O)c4cccc(O)c4[nH]c13)CC(C(C)(C)O)O2] | |

S-2: Disperse the collected cells in a solvent at a density of 5.0×10⁶ cells/mL-1.0×10⁷ cells/mL, and then subject them to sonication at 2° C.-8° C. to prepare a cell lysate, in which, the solvent is selected from any one or combination of physiological saline, 5% glucose solution, phosphate buffer solution (PBS), TBPS buffer, TBST buffer, Tris buffer;

S-3: separating the cell lysate prepared in step S-2, and then sequentially filtering a separated solution through 0.45 μm, 0.22 μm filter membrane, thereby obtaining the cell protein extract.

In the preferred technical solution of the present disclosure, the culture medium in step S-1 contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acid (18AA) 0.02-0.05%, and 3-8 μmol/L of stressors.

In the preferred technical solution of the present disclosure, the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05%, and 4-6 μmol/L of stressors.

In the preferred technical solution of the present disclosure, the density of mesenchymal stem cells in step S-1 is 6.0×10⁶-2.0×10⁷ cells/mL, preferably 8.0×10⁶-1.0×10⁷ cells/mL.

In the preferred technical solution of the present disclosure, the mesenchymal stem cells of step S-1 are cultured in a culture medium for 11-13 hours.

In the preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In the preferred technical solution of the present disclosure, the separation described in step S-1 is selected from any one or a combination of centrifugation and filtration, wherein conditions of the centrifugation are 1000-2000 rpm, 3-15 min, preferably 1200 rpm˜1500 rpm, 5-10 min.

In the preferred technical solution of the present disclosure, conditions of the sonication of step S-2 are: working at 2° C.-8° C., 25 kHz, 360 W for 3 seconds with a gap of 1 second, and sonication for 1-5 minutes.

In the preferred technical solution of the present disclosure, the separation described in step S-3 is selected from any one or a combination of 2000-8000 rpm, 10-30 min centrifugation, multi-stage centrifugation, and multi-stage filtration, preferably 3000-7000 rpm, 15-25 min.

In the preferred technical solution of the present disclosure, the multi-stage centrifugation in step S-3 is sequentially 3000-4000 rpm, 3-5 minutes, 5000-6000 rpm, 3-5 minutes, and 7000 rpm, 5-8 minutes.

In the preferred technical solution of the present disclosure, the pore size of the multi-stage filtration membrane is selected from any one of 80 μm, 50 μm, 30 μm, 10 μm, or 5 μm.

In the preferred technical solution of the present disclosure, the cell protein extract prepared in step S-3 or the protein composition prepared in step (2) is frozen, preferably frozen at −40° C. to −20° C.

In the preferred technical solution of the present disclosure, a freeze-drying protectant is added to the cell protein extract prepared in step S-3 or the protein composition prepared in step (2), and freeze-dry to obtain a cell protein extract freeze-dried preparation or protein composition freeze-dried preparation. The freeze-drying protectant is selected from any one or a combination of mannitol, sorbitol, dextran, glycerol, sucrose, trehalose, glucose, lactose, maltose, glucan, trioctylglycerol (HES), polyethylene glycol, ethylene glycol, phosphate, acetate, citrate, sorbitol, starch.

In the preferred technical solution of the present disclosure, the freeze-dried preparation contains a freeze-drying protectant of 0.5-8% by mass percentage, preferably 1-5%.

In the preferred technical solution of the present disclosure, a protein stabilizer is optionally added to the cell protein extract prepared in step S-3 or the protein composition prepared in step (2), wherein the protein stabilizer is selected from any one of albumin, zinc salt, or aluminum salt.

The preferred technical solution of the present disclosure, wherein a pH of the freeze-dried preparation of the cell protein extract or the freeze-dried preparation of the protein composition is 6-8, preferably 7-7.5.

In the preferred technical solution of the present disclosure, the cell protein extract prepared in step S-3 is hydrolyzed by either a nuclease or an omnipotent nuclease before separation and purification.

In the preferred technical solution of the present disclosure, the nuclease is selected from any one or a combination of RNA nuclease and DNA nuclease.

In the preferred technical solution of the present disclosure, 25 U/mL to 30 U/mL of any one of or a combination of a nuclease or an omnipotent nuclease is added to the cell protein extract prepared by the present disclosure, and it is subjected to enzymatic hydrolysis at 37° C.±1° C. for 20-30 minutes to obtain the enzymatic hydrolysate.

In the preferred technical solution of the present disclosure, the molecular weight of the protein composition having a joint repair function is 25 kDa to 245 kDa, preferably 50 kDa to 200 kDa.

The preferred technical solution of the present disclosure, wherein the protein constituents in the protein composition having a joint repair function is shown in FIG. 2.

In the preferred technical solution of the present disclosure, the freeze-dried preparation is remelted with an isotonic solution before use, and then used in any or a combination of application, rolling needle, microneedle, massage, intravenous injection, intramuscular injection, subcutaneous injection, acupoint injection, or lumbar puncture. The isotonic solution is selected from any or a combination of physiological saline, 5% glucose solution, phosphate buffer (PBS), TBPS buffer, TBST buffer, or Tris buffer.

The cultivation of mesenchymal stem cells or primary mesenchymal stem cells of the present disclosure adopts the cultivation methods in the art.

In the preferred technical solution of the present disclosure, the cultivation of the mesenchymal stem cells comprises the following steps: adding primary mesenchymal stem cells to a passaging medium with an initial density of 5.0×10⁵-5.0×10⁶ cells/mL, and then culturing them under conditions of 37.0° C.±0.5° C. and 5%±1.0% CO₂ for 10-15 days. Every 2-3 days, after observing the yellowing of the passaging medium, half of the passaging medium is replaced, wherein the passaging medium is DMEM/F12 medium containing 10% FBS, 100 U/mL penicillin, and 100 μg/mL streptomycin.

In the preferred technical solution of the present disclosure, the cultivation of the primary mesenchymal stem cells comprises the following steps:

A: After cleaning and disinfecting the umbilical cord, dissect the tissue, take the Wharton's Jelly tissue, cut it into 3 mm³ small pieces, centrifuge, clean, collect tissue blocks, place them in DMEM/F12 medium containing 10% fetal bovine serum FBS, 100 μg/mL penicillin, and 100 μg/mL streptomycin, and then culture them under 37.0° C.±0.5° C. and 5%±1.0% CO₂ conditions. Replace half of the culture medium every 2-3 days and continue the culture until the cells migrate out from the tissue blocks;

B: Shake and collect low-level cells, wash with PBS, add 0.25% trypsin for digestion for 2-3 minutes, add an equal volume of trypsin termination solution to stop digestion, gently blow with a straw, centrifuge at 1200-1500 rpm/min, 5-8 minutes, and collect the cells.

The purpose of the present disclosure is to provide a preparation method for a cell protein extract with joint repair efficacy, comprising the following steps:

S-1: Place mesenchymal passaged cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acid (18AA) 0.01-0.1%, and 2-10 μmol/L of a stressor, and then culture it under conditions of 37.0° C.±0.5° C. and 5%±1.0% CO₂ for 10 to 14 hours. The cells were separated, washed, and collected. The stressors are selected from any one of compounds 1-16 or a combination thereof;

S-2: Disperse the collected cells in the solvent at a density of 5.0×10⁶ cells/mL-1.0×10⁷cells/mL, and then sonicate at 2° C.-8° C. to prepare cell lysate, in which, the solvent is selected from any one or combination of physiological saline, 5% glucose solution, phosphate buffer solution (PBS), TBPS buffer, TBST buffer, Tris buffer;

S-3: separating the cell lysate prepared in step S-2, and then sequentially filtering a separated solution through 0.45 μm, 0.22 μm filter membrane, thereby obtaining the cell protein extract.

In the preferred technical solution of the present disclosure, the culture medium in step S-1contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acid (18AA) 0.02-0.05%, and 3-8 μmol/L of stressors.

In the preferred technical solution of the present disclosure, the culture medium in step S-1contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05%, and 4-6 μmol/L of stressors.

In the preferred technical solution of the present disclosure, the density of mesenchymal stem cells in step S-1 is 5.0 x106-2.0×10⁷ cells/mL, preferably 8.0×10⁶-1.0×10⁷ cells/mL.

In the preferred technical solution of the present disclosure, the mesenchymal stem cells of step S-1 are cultured in a culture medium for 11-13hours.

In the preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In the preferred technical solution of the present disclosure, the separation described in step S-1 is selected from any one or a combination of centrifugation and filtration, wherein the centrifugation conditions are 1000-2000 rpm, 3-15 min, preferably 1200 rpm˜1500 rpm, 5-10 min.

In the preferred technical solution of the present disclosure, conditions of the sonication of step S-2 are: working at 2° C.-8° C., 25 kHz, 360W for 3 seconds with a gap of 1 second, and sonication for 1-5 minutes.

In the preferred technical solution of the present disclosure, the separation described in step S-3 is selected from any one or a combination of 2000-8000 rpm, 10-30 min centrifugation, multi-stage centrifugation, and multi-stage filtration, preferably 3000-7000 rpm, 15-25 min.

In the preferred technical solution of the present disclosure, the multi-stage centrifugation in step S-3 is sequentially 3000-4000 rpm, 3-5 minutes, 5000-6000 rpm, 3-5 minutes, and 7000 rpm, 5-8 minutes.

In the preferred technical solution of the present disclosure, the pore size of the multi-stage filtration membrane is selected from any one of 80 μm, 50 μm, 30 μm, 10 μm, or 5 μm.

In the preferred technical solution of the present disclosure, the cell protein extract prepared in step S-3 is frozen, preferably at −40° C. to −20° C.

In the preferred technical solution of the present disclosure, the cell protein extract prepared in step S-3 is hydrolyzed by either a nuclease or an omnipotent nuclease before separation and purification.

The cultivation of mesenchymal stem cells or primary mesenchymal stem cells of the present disclosure adopts the cultivation methods in the art.

In the preferred technical solution of the present disclosure, the cultivation of the mesenchymal stem cells comprises the following steps: adding primary mesenchymal stem cells to a passaging medium with an initial density of 5.0×10⁵-5.0×10⁶ cells/mL, and then culturing them under conditions of 37.0° C.±0.5° C. and 5%±1.0% CO₂ for 10-15 days. Every 2-3 days, after observing the yellowing of the passaging medium, half of the passaging medium is replaced, wherein the passaging medium is DMEM/F12 medium containing 10% FBS, 100 U/mL penicillin, and 100 μg/mL streptomycin.

In the preferred technical solution of the present disclosure, the cultivation of the primary mesenchymal stem cells comprises the following steps:

A: After cleaning and disinfecting the umbilical cord, dissect the tissue, take the Wharton's Jelly tissue, cut it into 3 mm³ small pieces, centrifuge, clean, collect tissue blocks, place them in DMEM/F12 medium containing 10% fetal bovine serum FBS, 100 μg/mL penicillin, and 100 μg/mL streptomycin, and then culture them under 37.0° C.±0.5° C. and 5%±1.0% CO₂ conditions. Replace half of the culture medium every 2-3 days and continue the culture until the cells migrate out from the tissue blocks;

B: Shake and collect low-level cells, wash with PBS, add 0.25% trypsin for digestion for 2-3 minutes, add an equal volume of trypsin termination solution to stop digestion, gently blow with a straw, centrifuge at 1200-1500 rpm/min, 5-8 minutes, and collect the cells.

Another object of the present disclosure is to provide a preparation method for a protein composition having a joint repair function, comprising the following steps:

(1) Add any one of or a combination of a nuclease or an omnipotent nuclease of 20 U/mL-35 U/mL into a cell protein extract of the present disclosure, and place it under 37° C.±1° C. for enzymatic hydrolysis for 15-40 minutes to obtain the enzymatic hydrolysate, then place the enzymatic hydrolysate under 2° C.-4° C. conditions for backup;

(2) Under the conditions of 2° C.-8° C., the enzymatic hydrolysate prepared in step (1) was prepared with an eluent at a concentration of 5-15 mg/mL, and then passed through a chromatographic column with an eluent flow rate of 0.1-1 mL/min. Aneluent fraction with a UV wavelength of 280 nm is monitored and collected. The eluent is composed of 50 mmol/L phosphate buffer (pH 6.8) containing 300 mmol/L sodium chloride.

In the preferred technical solution of the present disclosure, the nuclease is selected from any one or a combination of RNA nuclease and DNA nuclease.

In the preferred technical solution of the present disclosure, 25 U/mL to 30 U/mL of any one of or a combination of a nuclease or an omnipotent nuclease is added to the cell protein extract prepared by the present disclosure, and it is subjected to enzymatic hydrolysis at 37° C.±1° C. for 20-30 minutes to obtain the enzymatic hydrolysate.

In the preferred technical solution of the present disclosure, the molecular weight of the protein composition having a joint repair function is 25 kDa to 245 kDa, preferably 50 kDa to 200 kDa.

In the preferred technical solution of the present disclosure, the protein composition obtained in step (2) is frozen, preferably at −40° C. to −20° C.

In the preferred technical solution of the present disclosure, a freeze-drying protectant is added to the protein composition collected in step (2), freeze-dry to obtain a protein composition freeze-dried preparation. The freeze-drying protectant is selected from any one or a combination of mannitol, sorbitol, dextran, glycerol, sucrose, trehalose, glucose, lactose, maltose, glucan, triglyceride (HES), polyethylene glycol, ethylene glycol, phosphate, acetate, citrate, sorbitol, starch.

In the preferred technical solution of the present disclosure, the freeze-dried preparation contains a freeze-drying protectant of 0.5-8% by mass percentage, preferably 1-5%.

In the preferred technical solution of the present disclosure, a protein stabilizer is optionally added to the protein composition collected in step (2), wherein the protein stabilizer is selected from any one of albumin, zinc salt, or aluminum salt.

The preferred technical solution of the present disclosure is a pH of the freeze-dried preparation of the protein composition is 6-8, preferably 7-7.5.

The preferred technical solution of the present disclosure, wherein the protein constituents in the protein composition having a joint repair function is shown in FIG. 2.

In the preferred technical solution of the present disclosure, the freeze-dried preparation is remelted with an isotonic solution before use, and then used in any or a combination of application, rolling needle, microneedle, massage, intravenous injection, intramuscular injection, subcutaneous injection, acupoint injection, or lumbar puncture. The isotonic solution is selected from any or a combination of physiological saline, 5% glucose solution, phosphate buffer (PBS), TBPS buffer, TBST buffer, or Tris buffer.

Another object of the present disclosure is to provide a joint repair composition, which is composed of any one or a combination of the cell protein extract with joint repair efficacy or the protein composition having a joint repair function of the present disclosure and a pharmaceutically acceptable carrier.

The pharmaceutically acceptable dosage or type of carrier of the present disclosure depends on factors such as the physicochemical properties and content of the active ingredients in the composition, formulation type, dissolution and bioavailability of the formulation.

The composition of the present disclosure can be various dosage forms in the field, and can be prepared using formulation techniques in the field.

In the preferred technical scheme of the disclosure, the composition is selected from any kind of freeze-dried agent, gel, nasal spray, liquid dressing, injection, patch, paste, cream, emulsion and suppository.

In the preferred technical solution of the present disclosure, the administration method of the combination is selected from any one or a combination of application, rolling needle, microneedle, massage, intravenous injection, muscle injection, subcutaneous injection, acupoint injection, lumbar puncture.

Another object of the present disclosure is to provide the cell protein extracts with joint repair efficacy, the protein compositions having a joint repair function, or their applications in the preparation of products for cell repair, joint repair, or cartilage repair.

In the preferred technical solution of the present disclosure, the joint repair or cartilage repair is selected from any one of traumatic arthropathy, degenerative osteoarthropathy, joint injury, refractory wound lesions, knee osteoarthrosis, or cartilage injury or their complications.

In the preferred technical solution of the present disclosure, the product is used for any one or a combination of bone and joint care populations, middle-aged and elderly bone degenerative disease populations, sports injury populations, sports maintenance populations, and postoperative rehabilitation populations for bone and joint diseases.

Another object of the present disclosure is to provide compounds 1-16 for the application of stress induced stem cell production of functional proteins with repair effects.

In the preferred technical solution of the present disclosure, the repair is any one of cell repair, hair follicle repair, joint repair, or repair.

Unless otherwise specified, when the present disclosure relates to the percentage between liquids, the said percentage is volume/volume percentage; When the present disclosure relates to the percentage between liquid and solid, the percentage is the volume/weight percentage; When the present disclosure relates to the percentage between solid and liquid, the percentage is the weight/volume percentage; The rest are weight/weight percentage.

Unless otherwise specified, the identification of mesenchymal stem cells (MSCs) in the present disclosure refers to Standards for the culture and quality control of umbilical cord mesenchymal stromal cells for neurorestorative clinical application»·

Compared with prior art, the present disclosure has the following beneficial effects:

1. The present disclosure scientifically screens culture media containing stressors to induce mesenchymal stem cells to produce cell proteins with cell repair effects. The obtained cell protein extracts, protein compositions, or their compositions have the effects of cell repair, joint repair, or cartilage repair, can activate wound chondrocyte regeneration, promote injury repair, and are used for the prevention and treatment of any one of traumatic arthropathy, degenerative osteoarthropathy, joint injury, refractory wound lesions, knee osteoarthrosis, or cartilage injury or their complications, and have the advantages of high purity, good stability, safety and effectiveness, and easy storage and transportation.

2. The preparation method of the present disclosure has the advantages of simple operation, green environmental protection, higher cost, and suitability for industrial production.

### BRIEF DESCRIPTION OF THE DRAWINGS

FIG. 1: Electrophoretic separation results of the protein composition having a joint repair function of the present disclosure;

FIG. 2 High performance liquid phase detection results of the protein composition having a joint repair function of the present disclosure;

FIG. 3: Study on the repair effect of left medial condylar joint injury in experimental sheep on day 0 and day 49 in Example 1;

FIG. 4: Study on the repair effect of right lateral platform joint injury in experimental sheep on day 0 and day 49 in Example 1.

FIG. 5: Clinical scoring results of the protein composition having a joint repair function of the present disclosure for repairing joint or cartilage injuries in patients;

FIG. 6: Functional score results of the protein composition having a joint repair function of the present disclosure for repairing joint or cartilage injuries in patients.

### DETAILED DESCRIPTION OF THE EMBODIMENTS

The following will further explain and describe the detailed content of the present disclosure in conjunction with specific embodiments, but this does not limit the scope of protection of the present disclosure.

### 1. Culture of Primary Mesenchymal Stem Cells

The cultivation of primary mesenchymal stem cells includes the following steps:

1) After cleaning and disinfecting the umbilical cord, dissect the tissue, take the Wharton's Jelly tissue, cut it into 3 mm³ small pieces, centrifuge, clean, collect tissue blocks, place them in a culture bottle, add DMEM/F12 culture medium containing 10% fetal bovine serum FBS, 100 μg/mL penicillin, and 100 μg/mL streptomycin, and then culture it under 37° C. and 5% CO₂ conditions to promote its adhesion. Observe the yellowing of the culture medium, and replace half of the culture medium every 2-3 days. Culture for 10-12 days until cells can be seen climbing out at the edge of the tissue block;

2) Gently shake to make the tissue block fall off, collect the tissue block and lower layer cells separately, and then attach the collected tissue block to the wall for culture;

3) After cleaning the collected low-level cells with PBS, add an appropriate amount of 0.25% trypsin and digest for 2-3 minutes. Add an equal volume of trypsin termination solution to stop digestion. Gently blow the bottom of the bottle with a straw, centrifuge at 1500 rpm/min. 5 minutes, and collect the cells.

### 2. Passage Culture of Primary Mesenchymal Stem Cells

Passage culture of primary mesenchymal stem cells: Primary mesenchymal stem cells were added to DMEM/F12 medium containing 10% FBS, 100 U/mL penicillin, and 100 μg/mL streptomycin at an initial density of 1.0×10⁵-6.0×10⁵ cells/mL, and then cultured under conditions of 37.0° C.±0.5° C. and 5%±1.0% CO₂ for 10-15 days, with intervals of 2-3 days. After observing the yellowing of the medium, half of the medium was replaced.

3. Reference 1 for the preparation of compounds 1-16 (New limonophyllines A-C from the stem of Atalantia monophylla and cytotoxicity against cholangiocarcinoma and HepG2 cell lines, Arch. Pharm. Res. (2018) 41:431-437).

### Example 1: Preparation of the Cell Protein Extract with Joint Repair Effect of the Present Disclosure

The preparation method of the cell protein extract with joint repair effect of the present disclosure includes the following steps:

Add mesenchymal passaged cells with a density of 5.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05% and compound 16 with 5 μmol/L; then, incubate it at 37° C. and 5% CO₂ for 30 minutes, then centrifuge it at 1200 rpm, 5 minutes, wash it with PBS three times, and collect the cells;

(2) Disperse the collected cells in step (1) into physiological saline at a density of 1.0×10⁷ cells/mL, and sonicate for 3 seconds with a gap of 1 second under conditions of 2-8° C., 25 kHz, and 360 W for 2 minutes to prepare cell lysate;

(3) Centrifuge the cell lysate prepared in step (2) at 7000 rpm, 20 minutes, and filter the obtained solution through 0.45 μm and 0.22 μm filter membranes sequentially to obtain the cell protein extract.

### Example 2: Preparation of the Protein Composition Having a Joint Repair Function of the Present Disclosure

The preparation of the protein composition having a joint repair function of the present disclosure includes the following steps:

(1) Add 25 U/mL of the omnipotent nuclease (UCF. ME UltraNuclease) to the cell protein extract prepared in Example 1, and incubate it at 37° C. for 30 minutes to obtain the enzymatic hydrolysate;

(2) Under the conditions of 2° C.-8° C., prepare the enzymatic hydrolysate prepared in step (1) with an eluent of 10 mg/mL, followed by a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), elution flow rate is 0.1-1 mL/min, and the elution fraction with a UV wavelength of 280 nm is monitored and collected. The eluent is composed of 50 mmol/L phosphate buffer (pH 6.8) containing 300 mmol/L sodium chloride.

### Example 3: Preparation of the Freeze-Dried Preparation of the Protein Composition Having a Joint Repair Function of the Present Disclosure

Add the required amount of mannitol to the protein composition prepared in Example 2, stir, mix well, and freeze dry. The resulting freeze-dried preparation contains 2% mannitol (m/m).

### Example 4: Preparation of the Cell Protein Extract with Joint Repair Effect of the Present Disclosure

The preparation method of the cell protein extract with joint repair effect of the present disclosure includes the following steps:

Add mesenchymal passaged cells with a density of 5.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05% and compound 13 with 8 μmol/L; then, incubate it at 37° C. and 5% CO₂ for 12 hours, then centrifuge it at 1200 rpm, 5 minutes, wash it with PBS three times, and collect the cells;

(2) Disperse the collected cells in step (1) into physiological saline at a density of 1.0×10⁷ cells/mL, and sonicate for 3 seconds with a gap of 1 second under conditions of 2-8° C., 25 kHz, and 360 W for 2 minutes to prepare a cell lysate;

(3) Centrifuge the cell lysate prepared in step (2) at 7000 rpm, 20 minutes, and filter the obtained solution through 0.45 μm and 0.22 μm filter membranes sequentially to obtain the cell protein extract with joint repair effect.

### Example 5: Preparation of the Joint Repair Protein Composition of the Present Disclosure

The preparation of the protein composition having a joint repair function of the present disclosure includes the following steps:

(1) Add 30 U/mL of the omnipotent nuclease (UCF. ME UltraNuclease) to the cell protein extract prepared in Example 4, and incubate it at 37° C. for 30 minutes to obtain the enzymatic hydrolysate;

(2) Under the conditions of 2° C.-8° C., prepare the enzymatic hydrolysate prepared in step (1) with an eluent of 10 mg/mL, followed by a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), elution flow rate is 0.1-1 mL/min, and the elution fraction with a UV wavelength of 280 nm is monitored and collected. The eluent is composed of 50 mmol/L phosphate buffer (pH 6.8) containing 300 mmol/L sodium chloride.

### Example 6: Preparation of the Freeze-Dried Preparation of the Protein Composition Having a Joint Repair Function of the Present Disclosure

Add dextran to the protein composition prepared in Example 5, stir well, mix well, and freeze dry. The resulting freeze-dried preparation contains 5% dextran (m/m).

### Example 7: Preparation of the Cell Protein Extract with Joint Repair Effect of the Present Disclosure

The preparation method of the cell protein extract with joint repair effect of the present disclosure includes the following steps:

Add mesenchymal passaged cells with a density of 6.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05% and compound 14 with 6 μmol/L; then, incubate it at 37° C. and 5% CO₂ for 12 hours, then centrifuge it at 1200 rpm, 5 minutes, wash it with PBS three times, and collect the cells;

(2) Disperse the collected cells in step (1) into physiological saline at a density of 9.0×10⁶ cells/mL, and sonicate for 3 seconds with a gap of 1 second under conditions of 2-8° C., 25 kHz, and 360 W for 2 minutes to prepare cell lysate;

(3) Centrifuge the cell lysate prepared in step (2) at 7000 rpm*20 minutes, and filter the obtained solution through 0.45 μm and 0.22 μm filter membranes sequentially to obtain the cell protein extract.

### Example 8: Preparation of the Protein Composition Having a Joint Repair Function of the Present Disclosure

The preparation of the protein composition having a joint repair function of the present disclosure includes the following steps:

(1) Add 20 U/mL of the omnipotent nuclease (UCF. ME UltraNuclease) to the cell protein extract prepared in Example 7, and incubate it at 37° C. for 30 minutes to obtain the enzymatic hydrolysate;

(2) Under the conditions of 2° C.-8° C., prepare the enzymatic hydrolysate prepared in step (1) with an eluent of 10 mg/mL, followed by a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), elution flow rate is 0.8 mL/min, and the elution fraction with a UV wavelength of 280 nm is monitored and collected. The eluent is composed of 50 mmol/L phosphate buffer (pH 6.8) containing 300 mmol/L sodium chloride.

### Example 9: Preparation of the Freeze-Dried Preparation of the Joint Repair Protein Composition of the Present Disclosure

Add sorbitol to the protein composition prepared in Example 8, stir well, mix well, and freeze dry. The resulting freeze-dried preparation contains 3% sorbitol (m/m).

### Example 10: Preparation of the Cell Protein Extract with Joint Repair Effect of the Present Disclosure

The preparation method of the cell protein extract with joint repair effect of the present disclosure includes the following steps:

Add mesenchymal passaged cells with a density of 1.0×10⁷ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acid (18AA) 0.05% and compound 15 with 7 μmol/L; then, incubate it at 37° C. and 5% CO₂ for 12 hours, then centrifuge it at 1200 rpm, 5 minutes, wash it with PBS three times, and collect the cells;

(2) Disperse the collected cells in step (1) into physiological saline at a density of 5.0×10⁷ cells/mL, and sonicate for 3 seconds with a gap of 1 second under conditions of 2-8° C., 25 kHz, and 360 W for 2 minutes to prepare cell lysate;

(3) Centrifuge the cell lysate prepared in step (2) at 7000 rpm, 20 minutes, and filter the obtained solution through 0.45 μm and 0.22 μm filter membranes sequentially to obtain the cell protein extract.

### Example 11: Preparation of the Protein Composition Having a Joint Repair Function of the Present Disclosure

The preparation of the protein composition having a joint repair function of the present disclosure includes the following steps:

(1) Add 30 U/mL of the omnipotent nuclease (UCF. ME UltraNuclease) to the cell protein extract prepared in Example 10, and incubate it at 37° C. for 30 minutes to obtain the enzymatic hydrolysate;

(2) Under the conditions of 2° C.-8° C., prepare the enzymatic hydrolysate prepared in step (1) with an eluent of 10 mg/mL, followed by a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), elution flow rate is 0.1-1 mL/min, and the elution fraction with a UV wavelength of 280 nm is monitored and collected. The eluent is composed of 50 mmol/L phosphate buffer (pH 6.8) containing 300 mmol/L sodium chloride.

### Example 12: Preparation of the Freeze-Dried Preparation of the Joint Repair Protein Composition of the Present Disclosure

Add mannitol to the protein composition prepared in Example 11, stir, mix well, and freeze dry. The resulting freeze-dried preparation contains 5% mannitol (m/m).

### Example 13: Preparation of the Freeze-Dried Cell Protein Extract Preparation of the Present Disclosure

Add the required amount of mannitol to the cell protein extract prepared in Example 1, stir, mix well, and freeze dry. The resulting freeze-dried preparation contains 2% mannitol (m/m).

### Example 14: Molecular Weight Distribution Detection of the Protein Composition Having a Joint Repair Function of the Present Disclosure

Using standard molecular weight polyacrylamide gel electrophoresis (PAGE) to detect the molecular weight distribution of the protein composition having a joint repair function of the disclosure, including the following steps:

1. Select a glass plate with a thickness of 1.5 mm and place it horizontally. Spread the adhesive solution (15% lower layer adhesive, 4% separating gel) prepared according to Table 1 onto the glass plate in sequence. Insert the comb vertically onto the lower layer adhesive;

TABLE 1

| | | | 15% Lower layer | |
|---|---|---|---|---|
| reagent | 4% Separating gel | | adhesive | |
| ddH2O | 6 | mL | 3.7 | mL |
| 30% | 1.33 | mL | 8 | mL |
| Acrylamide mix | | | | |
| solution | | | | |
| Tris | 2.5 mL (0.5M Tris pH 6.8) | | 4 mL (1.5M Tris pH 8.8) | |
| 10% SDS | 100 | μL | 160 | μL |
| 10% APS | 100 | μL | 160 | μL |
| TEMED | 10 | μL | 18 | μL |

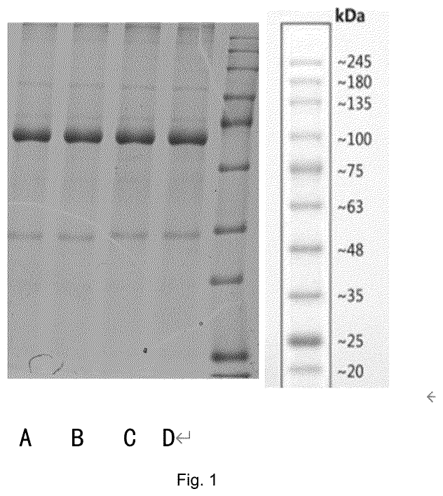

2. After thawing the frozen Marker (20 KDa-245 KDa) stored at −40° C., use PBS to prepare 10 μg/μL samples of the Marker and the freeze-dried preparation (freeze-dried powder) of the protein composition having a joint repair function of Embodiments 3, 6, 9 and 12, add 20 μL samples to the sample hole, electrophoresis at 60-80V until clearer bands appear, adjust the voltage to 100-120V until the Marker is completely separated, place the PAGE gel in the Coomassie brilliant blue dye solution, and stop dyeing when clear bands appear on the gel. After cleaning with pure water, decolorize with 10% acetic acid solution, and stop decolorizing when the gel becomes transparent. The results are shown in FIG. 1, where strip A is Example 3, strip B is Example 6, strip C is Example 9, and strip D is Example 12.

### Example 15: High Performance Liquid Chromatography Detection of the Joint Repair Protein Composition of the Present Disclosure

Dissolve the freeze-dried powder of the joint repair protein composition in deionized water in Example 3 and prepare it into a 10 mg/mL test solution.

Chromatographic column: SHIMSEN Ankylo (300 mm*4.6 mm.D., 3 μm; P/N: 380-01215-05) Shimadzu

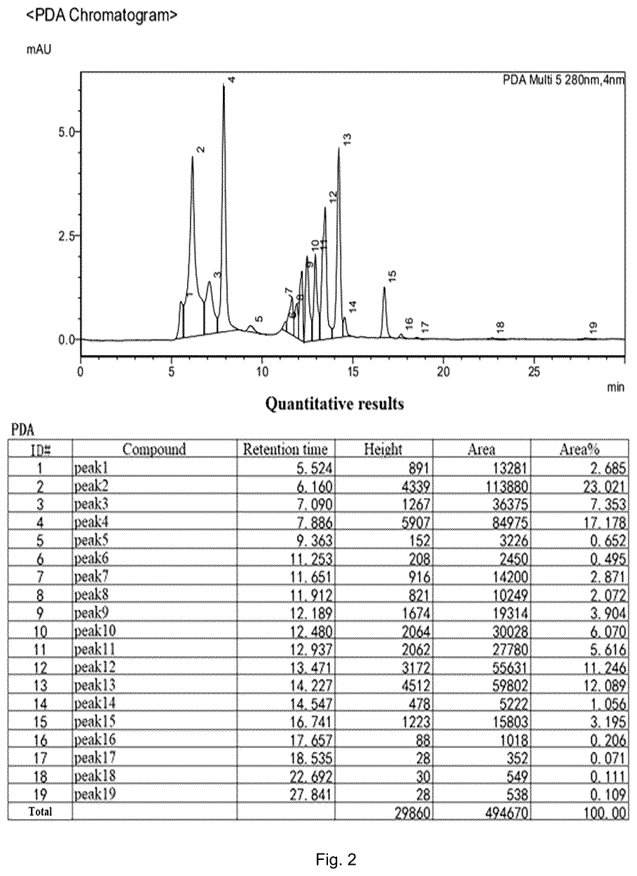

Mobile phase: 50 mmol/L phosphate buffer (pH-6.8), containing 300 mmol/L sodium chloride; Flow rate: 0.3 mL/min; Injection volume: 10 μL; Column temperature: 25° C.; Detection wavelength: 280 nm; Equal elution, collect for 30 minutes. The results are shown in FIG. 2.

### Experiment Example 1: Study on the Joint Repair Effect of the Cell Protein Extract with Joint Repair Effect of the Present Disclosure

Dissolve 120 mg of freeze-dried cell protein extract powder prepared in Example 13 in 5 mL of physiological saline to obtain a cell protein extract solution with a concentration of 2.4%. Prepare and use immediately before each injection.

### 1. Experimental Method

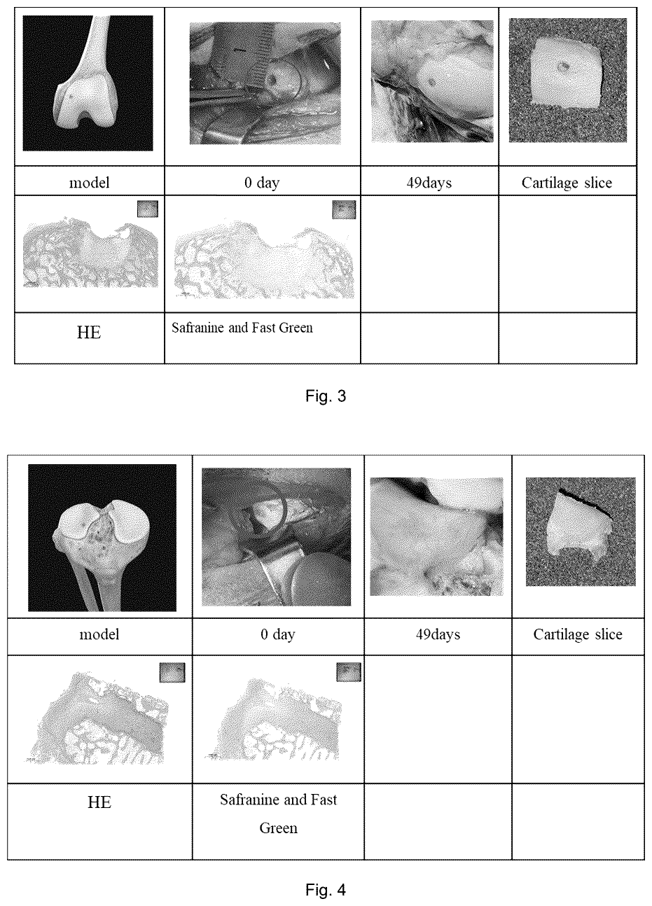

After general anesthesia, a 12 months old male goat weighing 47 kg was given a median incision of 8 cm on its left medial condyle. The medial edge of the patellar tendon was separated and cut open to the joint, exposing the femoral medial condyle and medial tibial plateau. The surface of the joint cartilage was smooth. A 3 mm ball drill was used to make a cartilage injury wound of 0.3×0.3×0.6 cm (length×width×depth) in the femoral medial condyle, and the incision was sutured (see FIG. 3). Take a median incision of 8 cm on its right lateral platform, separate layer by layer, and enter the joint capsule on the lateral side of the patellar tendon, exposing the femoral condyle and tibial lateral platform. The cartilage is intact and smooth. Use a 3 mm ball drill to make a cartilage injury wound of 0.6×0.3×0.3 cm (length×width×depth) on the lateral side of the platform, and suture the incision (see FIG. 4).

Inject 2.5 mL solution of the freeze-dried preparation of the protein composition having a joint repair function from Example 6 into the knee joint cavity of goats unilaterally every week for three consecutive weeks. Observe every morning and evening, and the results are shown in Table 2.

TABLE 2

| | |
|---|---|
| postoperative | Slightly restricted in activities, preferred to lie down rather than |
| | stand, and had a short standing time, with no abnormal diet. |
| postoperative | Could move, still unwilling to stand, spent more time lying |
| 0 D-7 D | down, and had no abnormal diet or mental state. |
| postoperative | Compared to before, standing time had significantly increased, |
| 8 D-14 D | walking activities had increased, and there were no |
| | abnormalities in diet and mental health. |
| postoperative | Standing was relatively normal compared to before, with normal |
| 15 D-21 D | activity and no abnormal diet or mental state. |
| postoperative | The activity and standing were basically normal without any |
| 21 D-28 D | abnormalities, and there were no abnormalities in diet, sleep, |
| | and mental state. |
| postoperative | There were no abnormalities in activity or standing, and there |
| 29 D-49 D | were no abnormalities in diet, sleep, or mental state. |

After 49 days of observation, the experimental sheep were anesthetized and euthanized, and cartilage sections were taken (see FIGS. 3-4).

This project had completed seven safety tests, including heat source detection, acute systemic toxicity test, cytotoxicity test, skin sensitization test, intradermal reaction test, local tissue reaction and degradation test after implantation, and subacute systemic toxicity test, and no adverse reactions were found.

The cell protein extract of the present disclosure has a superior repairing effect on joint injuries and is safe and effective.

### Experimental Example 2: Study on the Effect of the Protein Composition Having a Joint Repair Function of the Present Disclosure on Repairing Joint or Cartilage Injuries

Select 21 volunteer patients with joint lesions aged 50-80 as subjects (see Table 3) to investigate and evaluate the effect of the protein composition having a joint repair function of the present disclosure on repairing joint or cartilage injuries.

Subject inclusion criteria: [symbol] Patients diagnosed with knee osteoarthritis based on the American Association of Rheumatology (ACR) classification criteria; [symbol] Patients with knee joint pain caused by various factors that persist for more than half a year, or those who have improved after routine clinical treatment but have experienced recurrence or worsening of osteoarthritis after discontinuation of medication; the patient experiences pain in the knee joint for a score of at least 4 while walking on flat ground, which lasts for at least 4 weeks; [symbol] Through X-ray examination, it was observed that the Kellgren Lawrence grade of the target knee joint was grade II-III, while the Kellgren Lawrence grade of the other knee joint was lower than grade II; [symbol] 30-80 years old, regardless of gender; can complete four outpatient follow-up visits within six months (2 weeks, 4 weeks, 3 months, 6 months after treatment) after the first treatment; [symbol] Be able to understand and voluntarily sign informed consent forms, and be able to voluntarily complete trial procedures and follow-up examinations.

Exclusion criteria for subjects: 1) Secondary osteoarthritis, with a history and/or any evidence of the following diseases observed in the target joint: purulent arthritis, inflammatory joint disease, gout, recurrent pseudogout Paget's bone disease, joint fracture, melasma, acromegaly, hemochromatosis Wilson's disease, primary osteochondroma, hereditary diseases (such as ADHD), and collagen gene mutations; 2) Accompanied by other rheumatic diseases, including (but not limited to) systemic lupus erythematosus, inflammatory bowel disease Felty syndrome, scleroderma, inflammatory myopathy or other connective tissue diseases, overlap syndrome, etc.; 3) Individuals who have received intra-articular drug injections within 12 weeks; 4) Individuals who have undergone arthroscopy, corrective surgery, or total joint replacement surgery within 6 months; 5) Those who need to undergo arthroplasty; 6) Those who use anticoagulants (such as warfarin, low molecular weight heparin) and antiplatelet aggregation drugs; 7) Individuals with tumor diseases or a history of tumor diseases. 8) There are serious diseases under control, such as diabetes, hypertension, kidney disease, liver disease or serious heart disease (such as moderate and severe congestive heart failure (New York Heart Association cardiac function classification III/IV)), and the researcher judges that it is not suitable to join this study. 9) Abnormal laboratory test results: Blood routine: WBC<3×10\hat{ }9/L; HGB<90 g/L; PLT<100×10\hat{ }9/L; Liver function: TBIL>1.5 times the upper limit of normal value; ALT or AST>2.5 times the upper limit of normal value; Renal function: creatinine>1.5 times the upper limit of normal value, accompanied by creatinine clearance rate<50 mL/min (measured value, or calculated by Cockcroft Gault formula); HIVAb positive, HBsAg positive, HBcAb positive, HCVAb positive, syphilis antibody positive (excluding any positive test result). 10) Screening for individuals who have participated in clinical trials of other drugs within the first three months; 11) Within the first 4 weeks of screening, corticosteroids have been administered via intra-articular or intramuscular injection or intravenous injection; 12) Pregnant, breastfeeding, and pre pregnancy women. 13) Individuals who have used stem cell therapy within six months; 14) Individuals with mental disorders or uncontrolled and poorly controlled mental illnesses; 15) Those who are currently undergoing medical litigation; 16) Individuals with a history of alcohol and drug abuse, as well as those with allergies or a history of allergies; 17) The researchers consider that patients who are not suitable to participate in this clinical trial.

TABLE 3

| name | gender | age (year) | brief introduction of patient's condition |
|---|---|---|---|
| patient1 | female | 59 | Left knee joint pain has been present for more than five years. |
| | | | Two years ago, arthroscopic treatment was performed, but the |
| | | | pain still persists and affects mobility. |
| patient2 | female | 53 | Right knee joint pain for over five years without any history of |
| | | | treatment. |
| patient3 | female | 70 | Pain in both knees with limited function for 5 years, difficulty |
| | | | climbing stairs, and previous treatment with sodium hyaluronate |
| | | | injection in the knee joint five times, but still pain and limited |
| | | | mobility. |
| patient4 | female | 65 | Pain in both knees with limited mobility for 10 years, with ten |
| | | | injections of sodium hyaluronate into the joints, but still |
| | | | experiencing pain and limited mobility. |
| patient5 | female | 64 | Pain in the left and right knee joints with mild restricted |
| | | | mobility for 1 year, unable to walk for long periods of time. |
| patient6 | female | 67 | Pain in the left and right knee joints with limited mobility for 30 |
| | | | years, difficulty climbing stairs, and knee movements while |
| | | | walking. Previously treated with plaster and traditional Chinese |
| | | | medicine, but still experiencing pain and limited mobility. |
| patient7 | male | 78 | The right knee is painful when walking for 2 months and has |
| | | | limited mobility. It has been treated twice with a combination of |
| | | | traditional Chinese and Western medicine, with a duration of |
| | | | over 20 days per session. The therapeutic effect is good, but the |
| | | | pain and mobility are still limited. |
| patient8 | female | 64 | Occasional pain on both knees when going up and down stairs. |
| | | | Pain and tenderness appeared in the right knee more than 5 |
| | | | months ago, with limited mobility. |
| patient9 | male | 86 | Left knee treated with sodium hyaluronate for more than 10 |
| | | | years, stopped treatment more than 3 years ago, but still |
| | | | experiences pain and limited mobility. |
| patient10 | female | 56 | More than 10 years ago, both knees suffered from pain and |
| | | | prolonged walking. Despite taking painkillers and chondroitin, |
| | | | the patient still experienced pain and limited mobility. |
| patient11 | male | 80 | Pain in the right knee joint for over five years, difficulty |
| | | | climbing stairs, inability to walk for long periods of time, |
| | | | requiring crutches for assistance, and limited mobility. Injecting |
| | | | sodium hyaluronate ten times resulted in poor treatment effect, |
| | | | with continued pain and limited mobility in the right knee joint. |
| patient12 | female | 55 | More than 1 year ago, the left knee was swollen. have took |
| | | | synovitis granules and Gukang capsules, but still felt pain and |
| | | | limited mobility. |
| patient13 | female | 65 | More than 10 years ago, there was swelling and pain in the left |
| | | | knee, and two minimally invasive surgeries were performed. |
| | | | Recurrent one year later, with pain in the left knee joint and |
| | | | limited mobility. |
| patient14 | female | 67 | More than 8 years ago, there was swelling and pain in the right |
| | | | knee, MRI patient has accumulated fluid and has taken |
| | | | aminoglucose before, but still experiences pain and limited |
| | | | mobility. |
| patient15 | male | 55 | Right knee joint pain with limited mobility for more than 6 |
| | | | months, treated with oral analgesics, but still experiencing pain |
| | | | and limited mobility. |
| patient16 | female | 67 | Pain in both knee joints with limited mobility for more than 7 |
| | | | months. |
| patient17 | male | 58 | The left knee joint was painful with limited movement for 3 |
| | | | days, and occasionally there was a bounce. The effect of topical |
| | | | plaster and acupuncture and moxibustion treatment was not |
| | | | good. The right knee joint had been injected with stem cells for |
| | | | treatment, but it was still painful and movement was limited. |
| patient18 | male | 52 | Bilateral knee joint pain and limited mobility for more than 5 |
| | | | months, during which medication and rehabilitation treatment |
| | | | were performed, but the pain and movement were still limited. |
| patient19 | female | 66 | More than 10 years ago, there was experiencing knee pain while |
| | | | going up and down stairs and doing household chores. had joint |
| | | | effusion in the outpatient department and had undergone small |
| | | | needle surgery and physical therapy. The effusion disappeared, |
| | | | but still felt pain and limited mobility. |
| patient20 | male | 64 | Right knee joint pain for 5 years, worsened after activity and |
| | | | limited mobility. |
| patient21 | female | 64 | More than five years ago, there was knee pain when went |
| | | | upstairs and downstairs. It was diagnosed as degenerative |
| | | | disease of the knee joint. Had applied medicine, massage and |
| | | | acupuncture and moxibustion for 2 months, and the effect was |
| | | | fair. Occasionally took Fenbid, but it was still painful, with |
| | | | limited movement and squatting difficulty. |

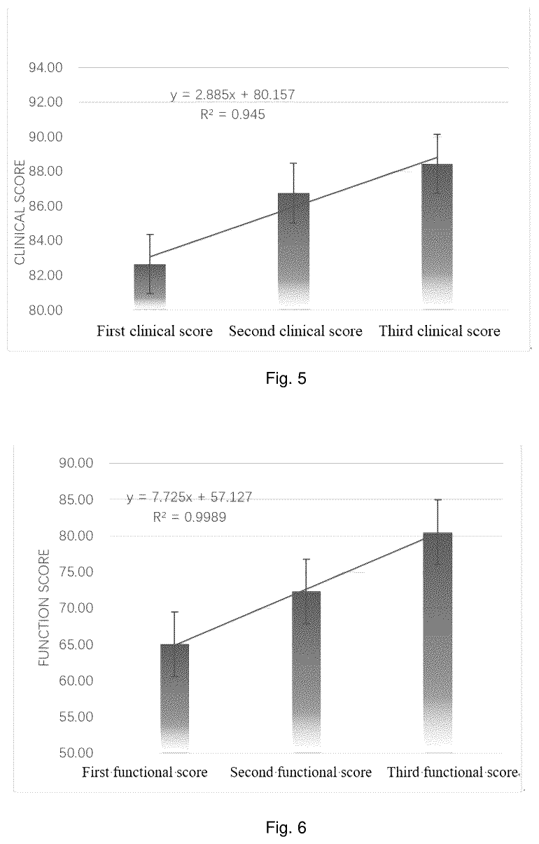

The knee joint function score (KSS) (Keen Society Score) (see Table 4) was used to observe and evaluate the therapeutic effect of drugs. Dissolve 65 mg of freeze-dried preparation of the protein combination powder having a joint repair function from Example 3 in 5 mL of physiological saline, and inject it into the unilateral knee joint cavity of the subject once a week, three times per course of treatment. The results are shown in FIGS. 5-6. The joint repair protein composition of the present disclosure can significantly repair joint or cartilage injuries in patients with joint or cartilage injuries.

TABLE 4

| About chief | Walking flat | | | |
|---|---|---|---|---|
| complaint | Painless (35 points), mild or occasional pain (30 points), moderate pain (15 points), | | | |
| pain | Severe pain (0 points) | | | |
| (50 points) | Climbing stairs | | | |
| | Painless (15 points), mild or occasional pain (10 points), moderate pain (5 points), | | | |
| | Severe pain (0 points) | | | |
| Stability | Internal and external displacement | | | |
| (25 points) | <5 mm(15 points), 6~9 mm(10 points), 10~14 mm(5 points), >15 mm(0 points) | | | |
| | Forward and backward displacement | | | |
| | <5 mm(10 points), 5~10 mm(5 points), >10 mm(0 points) | | | |
| Activity | The scoring standard is 1 point per 5° | | | |
| scope (25 | | | | |
| points) | | | | |
| Defects | Overextension | | | |
| (points | No overextension (0 points), <10°(−5 points), 10°~20°(−10 points), >20°(−15 points) | | | |
| deducted) | Flexion contracture | | | |
| | <5°(0 points), 6°~10°(−2 points), 11°~15°(−5 points), 16°~20°(−10 points), >20°(−15 | | | |
| | points) | | | |
| | Force line deformity | | | |
| | 5°~10°(0 points), every 5° increase in inversion/extraversion (−3 points) | | | |
| | Pain during rest | | | |
| | Mild pain (−5 points), Moderate Pain (−10 points), severe pain (−15 points) | | | |
| Clinical | Clinical total score A + B + C − D = Points Excellent □ Good □ | | | |
| Score | OK □ Poor □ | | | |
| Function | A. Walking condition (50 points) | | | |
| score | No Limit (50 points) The continuous walking distance exceeds 2 kilometers | | | |
| | (40 minutes). | | | |
| | The continuous walking distance ranges from 1 to 2 kilometers. (30 points) | | | |
| | continuous walking distance less than 1 kilometer (20 points) | | | |
| | can only move indoors (10 points) cannot walk (0 points) | | | |
| | B. Climbing Stairs condition (50 points) | | | |
| | Normal up and down stairs (50 points) normal up and down stairs, with the help | | | |
| | of handrails (40 points), need to use handrails to get up and down stairs (30 points) | | | |
| | can go up the stairs with the help of handrails, but cannot go down the stairs | | | |
| | independently (15 points) cannot go up and down the stairs at all (0 points) | | | |
| | C. Functional defects (points deducted) | | | |
| | Walk with a single stick (−5 points) Walking with two sticks (−10 points) | | | |
| | Support Activities with armpit staff or walking aids (−20 points) | | | |
| | Function total score A + B − C = Points (If the total score is negative, the score | | | |
| | is 0) | | | |
| | Excellent □ Good □ OK □ Poor □ | | | |
| Additional | Actual activity scope | Buckling | Straighten | |
| items | Muscle strength | Knee flexion | knee extension | |
| | deformity | Extraversion | inversion | Flexion contracture |

deformity

The above description of the specific embodiments of the present disclosure does not limit the present disclosure. Those skilled in the art may make various changes or deformations based on the present disclosure, as long as they do not deviate from the spirit of the present disclosure, they should fall within the scope of protection of the claims of the present disclosure.

1. A protein composition having a joint repair function, comprising the following steps for preparation:
(1) adding any one of or a combination of a nuclease or an omnipotent nuclease of 20 U/mL-35 U/mL into a cell protein extract, and performing enzymatic hydrolysis at 37° C.±1° C. for 15-40 minutes to obtain an enzymatic hydrolysate, then placing the enzymatic hydrolysate under 2° C.-4° C. conditions for backup;
(2) under the conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluent fraction with a UV wavelength of 280 nm, wherein the eluent consists of 50 mmol/L phosphate buffer (pH 6.8) comprising 300 mmol/L sodium chloride.

2. The protein composition as claimed in claim 1, wherein a molecular weight of the protein composition having a joint repair function is 20 kDa to 300 kDa, preferably 50 kDa to 200 kDa.

3. The protein composition as claimed in claim 1, wherein a preparation of the cell protein extract comprises the following steps:
S-1: placing mesenchymal passaged cells with a density of 5.0×10⁶ cells/mL to 1.0×10⁷ cells/mL in a culture medium comprising DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acid (18AA) 0.01-0.1%, and 2-10 μmol/L of a stressor, and then culturing the cells under conditions of 37.0° C.±0.5° C. and 5%±1.0% CO₂ for 10 to 14 hours, then separating, washing, and collecting the cells, and the stressor is selected from any one of compounds 1-16 or a combination thereof;

| Compounds | General formula | Substituents |
|---|---|---|
| 1 2 3 | 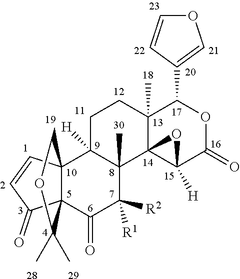 | R¹ = OCH₃, R² = OH R¹ = R² = OH R¹ = OH, R² = H |
| 4 5 | 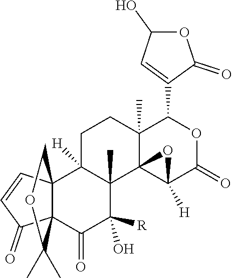 | R = H R = OH |
| 6 7 8 9 10 11 12 | 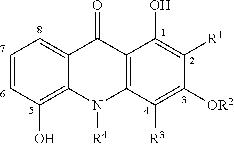 | R¹ = prenyl, R² = H, R³ = prenyl, R⁴ = CH₃ R¹ = prenyl, R² = H, R³ = prenyl, R⁴ = H R¹ = H, R² = H, R³ = OCH₃, R⁴ = CH₃ R¹ = H, R² = CH₃, R³ = OCH₃, R⁴ = CH₃ R¹ = OCH₃, R² = H, R³ = H, R⁴ = CH₃ R¹ = OCH₃, R² = H, R³ = OCH₃, R⁴ = CH₃ R¹ = prenyl, R² = CH₃, R³ = H, R⁴ = H |
| 13 14 | 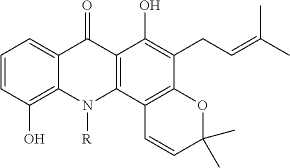 | R = H R = CH₃ |
| 15 | 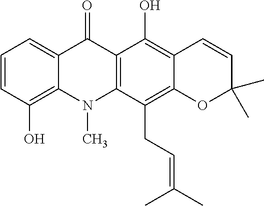 [SMILES: CC(C)=CCc1c2c(c(O)c3c(=O)c4cccc(O)c4n(C)c13)C=CC(C)(C)O2] | |
| 16 | 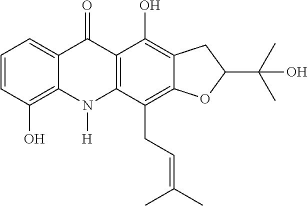 [SMILES: CC(C)=CCc1c2c(c(O)c3c(=O)c4cccc(O)c4[nH]c13)CC(C(C)(C)O)O2] | |

S-2: dispersing the collected cells in a solvent at a density of 5.0×10⁶ cells/mL-1.0×10⁷ cells/mL, and then obtaining a cell lysate by sonicating at 2° C.-8° C., in which, the solvent is selected from any one or combination of physiological saline, 5% glucose solution, phosphate buffer solution (PBS), TBPS buffer, TBST buffer, Tris buffer;
S-3: separating the cell lysate prepared in S-2, and then sequentially filtering a separated solution through 0.45 μm, 0.22 μm filter membrane, thereby obtaining the cell protein extract.

4. The protein composition as claimed in claim 1, wherein culturing the mesenchymal passage cells of step S-1 in a culture medium for 11-13 hours.

5. The protein composition as claimed in claim 1, wherein the separating of S-1 is selected from any one or a combination of centrifugation and filtration, wherein conditions of the centrifugation are 1000-2000 rpm, 3-15 min, preferably 1200 rpm˜1500 rpm, 5-10 min.

6. The protein composition as claimed in claim 1, wherein conditions of the sonicating of S-2 are: operating at 2° C.-8° C., 25 kHz, 360 W for 3 seconds with a gap of 1 second, and sonicating for 1-5 minutes.

7. The protein composition as claimed in claim 1, wherein adding a freeze-drying protectant to the eluent fraction collected in step (2) and freeze-drying and obtaining a freeze-dried preparation of the protein composition, wherein the freeze-drying protectant is selected from any one or a combination of mannitol, sorbitol, dextran, glycerol, sucrose, trehalose, glucose, lactose, maltose, glucan, trioctylglycerol, polyethylene glycol, phosphate, acetate, citrate, starch.

8. The protein composition as claimed in claim 1, wherein a pH of the freeze-dried preparation of the protein composition is 6-8, preferably 7-7.5.

9. A joint repair composition comprising the composition having a joint repair function as claimed in claim 1 and a pharmaceutically acceptable carrier.

10. Application of the protein composition having a joint repair function as claimed in claim 1 in the preparation of products for cell repair, joint repair, or cartilage repair.
